(19) United States
(12) Patent Application Publication
(10) Pub. No.: US 2013/0023001 A1
(43) Pub. Date: Jan. 24, 2013

(54) USE OF A SUBSTRATE IN A METHOD FOR MEASURING THE ACTIVITY OF PROTEOLYTIC ENZYMES

(71) Applicants: Gérard QUENTIN, Asnieres sur Seine (FR); Audrey CARLO, Asnieres sur Seine (FR); Loïse BOULANGER, Asnieres sur Seine (FR)

(72) Inventors: Gérard QUENTIN, Asnieres sur Seine (FR); Audrey CARLO, Asnieres sur Seine (FR); Loïse BOULANGER, Asnieres sur Seine (FR)

(21) Appl. No.: 13/553,920

(22) Filed: Jul. 20, 2012

(30) Foreign Application Priority Data
Jul. 22, 2011 (FR) 1156715

(51) Int. Cl. C12Q 1/56

(52) U.S. Cl. 435/13

(57) ABSTRACT

The disclosure relates to the use of a proteolytic enzyme substrate of general formula: Q¹-Xaa²-Xaa¹-rhodamine₁₁₀-Q², in which Xaa¹ and Xaa² are amino acids and Q² is Xaa¹-Xaa²-Q¹, or an amino acid Xaa³, or a group comprising an aryl group, or H, so as to be able to inhibit, in a blood sample containing a glycosaminoglycan, the anticoagulant capacity of said glycosaminoglycan. The invention also relates to a method for measuring an enzymatic activity using such a substrate and a kit for implementation thereof.

The present invention relates to the use of a proteolytic enzyme substrate, in particular in a method for measuring the activity of proteolytic enzymes in a blood sample. The invention also relates to a measuring kit which makes it possible to implement said method. More particularly, the method aims to measure, in a blood sample belonging to a patient treated with an anticoagulant, the activity of the enzymes responsible for hemostasis. This physiological process, which aims to interrupt bleeding, involves a cascade of enzymes and of coagulation factors. The anticoagulant in question is a molecule of the glycosaminoglycan family and more particularly a heparin. A distinction will be made between high-molecular-weight heparins termed: “unfractionated heparins” or abbreviated to UFH, and heparins termed: “low-molecular-weight heparins” or abbreviated to LMWH. The various heparins are administered to patients according to their pathological condition.

Usually, measuring methods aim to test the activity of enzymes, for example thrombin or plasmin. The enzyme substrates used in these measuring methods are then formed from a synthetic peptide consisting of two or three amino acids having an affinity for the enzyme and of a fluorophore group bonded to said peptide. The enzyme is then capable of cleaving the substrate between the fluorophore and the peptide, said fluorophore changing state and then being capable of providing a detectable fluorescent signal. Thus, the substrate is also referred to as a tracer reagent.

Reference may be made to document EP 1 833 982 which describes, for example, a thrombin-specific enzyme substrate which has a tripeptide coupled to 7-amino-4-methylcoumarin, commonly abbreviated to AMC. It also describes a plasmin-specific enzyme substrate which has a bis-dipeptide coupled to rhodamine₁₁₀.

According to the usual methods for measuring the activity of proteolytic enzymes, a blood sample, of blood or plasma, is provided and brought into contact with an activator, for example tissue factor, which will make it possible to activate the production of the enzyme. Next, the substrate, or tracer, is provided with an initiator, for example calcium in ionic form. The blood sample and the activator are then brought into contact with the substrate and the initiator in the same medium in which, on the one hand, the coagulation reactions and, on the other hand, the reaction of the enzyme with the substrate will take place. The initiator will make it possible to initiate the coagulation process, while the activator induces the production of the enzyme.

In addition, it is necessary to neutralize the glycosaminoglycans contained in the blood sample during the measurement of the activity, in order to dispense with their effect on the coagulation process. To do this, a synthetic polymer is provided which is formed from quaternary ammonium units linked via hydrocarbon-based chains of various lengths, for example polybrene or hexadimethrine, and which makes it possible to neutralize the effects of the glycosaminoglycans in the blood sample.

These synthetic polymers are introduced into the medium, either with the substrate or with the activator. Although they are effective, they are not easy to solubilize and require many steps before being homogeneously dispersed with the substrate or the activator. The complexity of the composition of the measuring kits is accordingly increased, and consequently so are the development and production costs.

In addition, the synthetic polymers accentuate the turbidity of the medium, such that the optical measurement of the fluorescent signal emitted by the fluorophore is disrupted. Consequently, the measurement of the activity of the enzyme contains an imprecision.

Thus, a problem which arises and which the present invention aims to solve is that of using a proteolytic enzyme substrate which not only can be used more easily and less expensively, but also makes it possible to lift this imprecision regarding the measurement of the activity.

With the aim of solving this problem, and according to a first subject, the present invention proposes the use of a proteolytic enzyme substrate of general formula: Q¹-Xaa²-Xaa¹-rhodamine₁₁₀-Q², in which: Xaa¹ and Xaa² are amino acids; and Q² is Xaa¹-Xaa²-Q¹, where an amino acid Xaa³, or a group comprising an aryl group, or else H; so as to able to inhibit, in a blood sample containing a glyco-saminoglycan, the anticoagulant capacity of said glycosaminoglycan.

Thus, by virtue of this proteolytic enzyme substrate, which makes it possible to measure the activity of the enzyme by means of its fluorophore, rhodamine₁₁₀, the glycosamino-glycans contained in the blood sample are also and simultaneously neutralized. In that way, this proteolytic enzyme substrate is able to play both the role of tracer reagent and that of an agent for neutralizing glycosaminoglycans. Thus, the difficult use of synthetic polymers aimed at neutralizing the glycosaminoglycans is thus dispensed with, and consequently, the costs of preparing the measuring kits or packs are reduced, as will be explained in greater detail hereinafter.

Furthermore, the proteolytic enzyme substrate which is the subject of the invention has no impact on the turbidity of the medium. Consequently, the optical measurement of the fluorescence is in no way disrupted when the enzyme cleaves the substrate between the rhodamine and the peptide.

It will be observed that the proteolytic enzymes targeted are those of the serine protease family, for example thrombin, plasmin, factor Xa or else activated protein C. These examples of proteolytic enzymes are not of course limiting.

According to one particularly advantageous embodiment of the invention, Q¹ is H; or the R¹O—(C═O)—CH₂—CO group in which R¹ comprises an alkyl or aryl group; or the R²—Xaa⁴ group in which Xaa⁴ is an amino acid and R² is a protective group or H. In that way, an enzyme substrate exhibiting good solubility is obtained, and consequently a homogeneous reactive medium. The fluorescence measurements are thus reproducible.

Advantageously, Xaa¹ is a basic amino acid, for example arginine. In that way, the substrate offers perfect recognition of the enzyme.

According to one particular embodiment of the invention, Q² is Xaa¹-Xaa²-Q¹, while Q¹ is the R^{2l —Xaa}⁴ group, Xaa² and Xaa⁴ being respectively glycine, so as to confer on said substrate a low affinity with respect to the proteolytic enzymes. The substrate with such a structure makes it possible to more easily measure the monitoring of the appearance of an enzymatic activity, more commonly called “generation”. This is because in fact, in the case of measurements of this type, it is important to take into account the whole of the cascade of enzymatic reactions and not to disrupt their kinetics. Since the substrate has a lower affinity with respect to the enzymes, the variation in the concentration of enzymes mobilized by the hydrolysis of the substrate will therefore be negligible and will have a very small impact on the rate of the other enzymatic reactions of the cascade. Although the reaction of the substrate which is the subject of the invention and of the enzyme under consideration has a relatively fragile rate constant, the fluorescent signal will nevertheless be considerable and detectable since rhodamine₁₁₀ exhibits a large amount of fluorescence. In addition, this fluorophore has excitation and emission wavelengths which are shifted with respect to the 7-amino-4-methylcoumarin normally used for this type of measurement, and which do not interfere with the hemoglobin possibly present in the blood sample.

According to another particular embodiment, in which Q² is Xaa¹-Xaa²-Q¹, while Q¹ is the R¹O—(C═O)—CH₂—CO group, Xaa² being valine and R¹ the ethyl group, a low affinity with respect to proteolytic enzymes is conferred on said substrate. Consequently, the appearance of an enzymatic activity is also measured without disrupting the kinetics of the enzymatic reaction cascade.

According to one variant of embodiment of the invention which is particularly advantageous, the amino acid Xaa³ is arginine. In that way, and as will be explained in greater detail in the remainder of the description, in particular when Xaa¹ is also arginine, there is total neutralization of the glycosaminoglycans, heparin.

According to another variant of embodiment of the invention, when Q² is a group comprising an aryl group, Q² comprises a —(C═O)ₘ—(O)ₙ— group linked to rhodamine₁₁₀, in which n=0 or 1, and m=0 or 1. In that way, the substrate is easier to produce. Indeed, by virtue of the —(C═O)ₙ—(O)ₙ— group, the attachment of the aryl group to rhodamine₁₁₀ is facilitated. Advantageously, n=0 and m=1, and the group is simply a carbonyl function. For example, Q² is the benzoyl group, i.e. a benzene ring, linked to rhodamine₁₁₀ via the carbonyl function, or else the 2-phenylbutanoyl group.

In addition, according to said other embodiment variant, Q² advantageously comprises a heteroatom, for example a nitrogen atom. The basic nature of this heteroatom may make it possible to reinforce the neutralization of the heparin.

According to another subject, the present invention proposes a method for measuring the activity of proteolytic enzymes of a blood sample, said method being of the type according to which: a blood sample which contains proteolytic enzymes and which may contain a glycosaminoglycan is provided; a proteolytic enzyme substrate capable of providing a signal when said proteolytic enzymes react with said substrate is provided; said blood sample is brought into contact with said proteolytic enzyme substrate, while the anticoagulant capacity of said glycosaminoglycan is inhibited so as to allow said proteolytic enzymes to react freely with said substrate in order to provide a signal representative of the activity of the proteolytic enzymes in said sample; and according to the invention, a proteolytic enzyme substrate of general formula: Q¹-Xaa²-Xaa¹-rhodamine₁₁₀-Q², in which: Xaa¹ and Xaa² are amino acids; and Q² is Xaa¹-Xaa²-Q¹, or an amino acid Xaa³, or a group comprising an aryl group, or H, is used so as to be able both to inhibit the anticoagulant capacity of the glycosaminoglycan and to measure the activity of the proteolytic enzymes, with a single compound. The proteolytic enzyme substrate used includes all the abovementioned variants and embodiments.

Preferentially, an activating reagent which makes it possible to induce the generation of proteolytic enzymes is also provided, and said activating reagent is brought into contact with said blood sample and said proteolytic enzyme substrate. As soon as the activating reagent comes into contact with the blood sample, it causes the generation of the proteolytic enzymes. For example, said activating reagent is chosen from tissue factor, phospholipids, thromboplastin, kaolin, ellagic acid, collagen, adenosine diphosphate, arachidonic acid, a thrombin receptor-activating peptide, or a combination thereof. Advantageously, an activator which is capable of acting a long way upstream in the coagulation and fibrinolysis enzymatic reaction cascade will be chosen, in particular when the measurement is aimed at monitoring the appearance of an enzymatic activity. The use of the substrate which is the subject of the invention is therefore advantageous as an agent for neutralizing heparin, since the latter acts directly or indirectly on most of the enzymes of the enzymatic cascade.

In addition, an initiating reagent for initiating the proteolytic enzyme reactions is provided, and said initiating reagent is brought into contact with said blood sample and said proteolytic enzyme substrate. In that way, the initiator will make it possible to initiate the coagulation process, while the activator brings about the generation of the enzymes. Advantageously, said initiating reagent is calcium, and it is provided, for example, in the form of calcium citrate.

According to one embodiment of the measuring method in accordance with the invention, said blood sample is brought into contact with said proteolytic enzyme substrate at a substrate concentration of between 50×10⁻⁶ and 1000×10⁻⁶ mol.L⁻¹, for example between 300×10⁻⁶ and 500×10⁻⁶ mol.L⁻¹. In that way, effective neutralization of the glycosaminoglycans is achieved and also an optical signal making it possible to measure the enzymatic activity is obtained.

According to yet another subject, the present invention proposes a kit for measuring the activity of proteolytic enzymes of a blood sample, for implementing the measuring method described above. According to the invention, the kit comprises: an activating reagent for inducing the generation of proteolytic enzymes; a proteolytic enzyme substrate of general formula Q¹-Xaa²-Xaa¹-rhodamine₁₁₀-Q², in which: Xaa¹ and Xaa² are amino acids; and Q² is Xaa¹-Xaa²-Q¹, or an amino acid Xaa³, or a group comprising an aryl group, or H; and an initiating reagent for initiating the enzymatic reactions. The proteolytic enzyme substrate used includes all the abovementioned variants and embodiments.

According to a first embodiment variant of the kit, said proteolytic enzyme substrate is mixed with said initiating reagent, in the same container, whereas the activating reagent is contained in another container. The enzyme substrate is then, for example, at a concentration of between 100×10⁻⁶ mol.L⁻¹ and 500×10⁻⁶ mol.L⁻¹, while the initiating reagent is at a concentration of between 10×10⁻³ mol.L⁻¹ and 20×10⁻³ mol.L⁻¹. The activating reagent is itself at a concentration of between, for example, 0.5×10⁻¹² and 2×10⁻¹² mol.L⁻¹.

According to a second variant, said activating reagent is mixed with said initiating reagent in the same container, whereas the enzyme substrate is contained alone in another container. This second embodiment variant makes it possible in particular not to disrupt the stability of the substrate.

When the kit which is the subject of the invention is used, firstly 40 μL of a blood sample for example, and secondly 10 μL of enzyme substrate, alone or mixed with the initiating reagent, and 10 μL of the activating reagent, respectively mixed with the initiating reagent or alone, are provided. These sample and reagent volumes are of course indications, and are adjusted according to the measuring devices and also to the types of measurement. They can just as easily be multiples thereof such as 80 μL/20 μL/20 μL, or else other volumes and relative proportions, for example 30 μL/15 μL/15 μL.

Other particularities and advantages of the invention will emerge on reading the description provided hereinafter of particular embodiments of the invention, given by way of nonlimiting indication, with reference to the appended drawings in which:

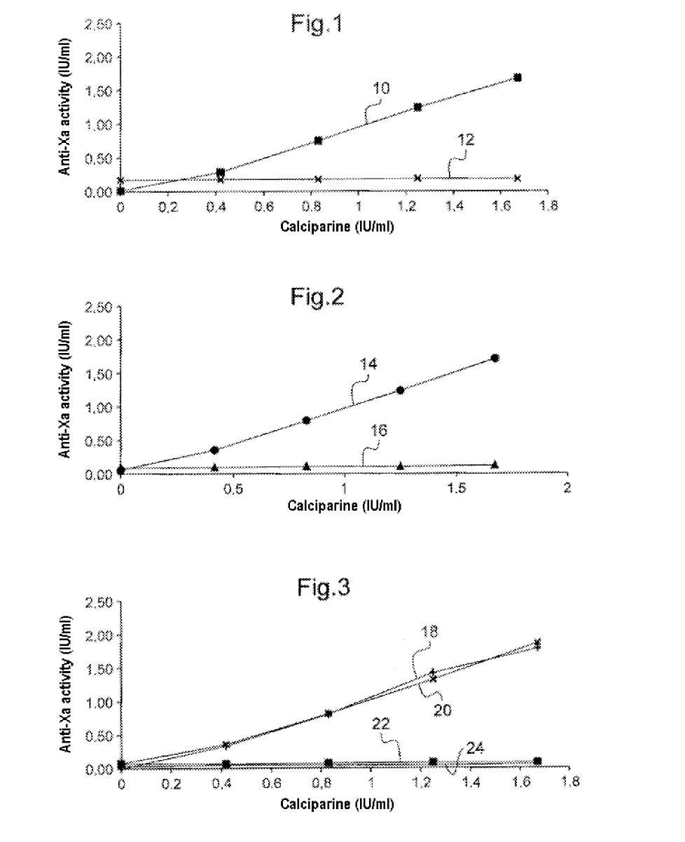

FIG. 1 is a graph showing enzymatic activities as a function of a heparin concentration according to a first example of implementation of an evaluation method;

FIG. 2 is a graph showing enzymatic activities as a function of a heparin concentration according to a second example of implementation of an evaluation method;

FIG. 3 is a graph showing enzymatic activities as a function of a heparin concentration according to a third example of implementation of an evaluation method;

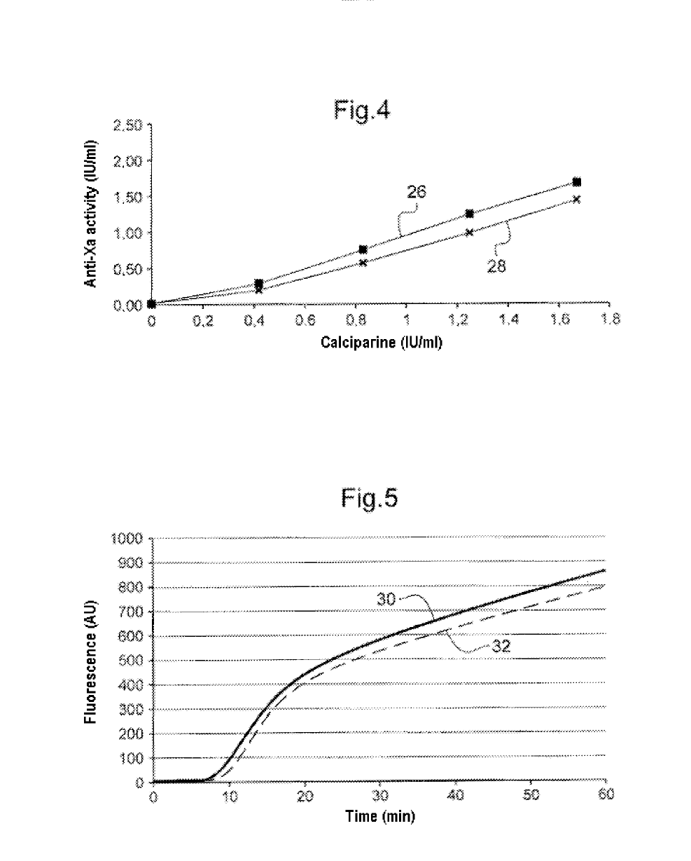

FIG. 4 is a graph showing enzymatic activities as a function of a heparin concentration according to a fourth example of implementation of an evaluation method;

FIG. 5 is a graph showing the strength of a fluorescent signal as a function of time according to the measuring method in accordance with the invention, with an unfractionated heparin at various concentrations;

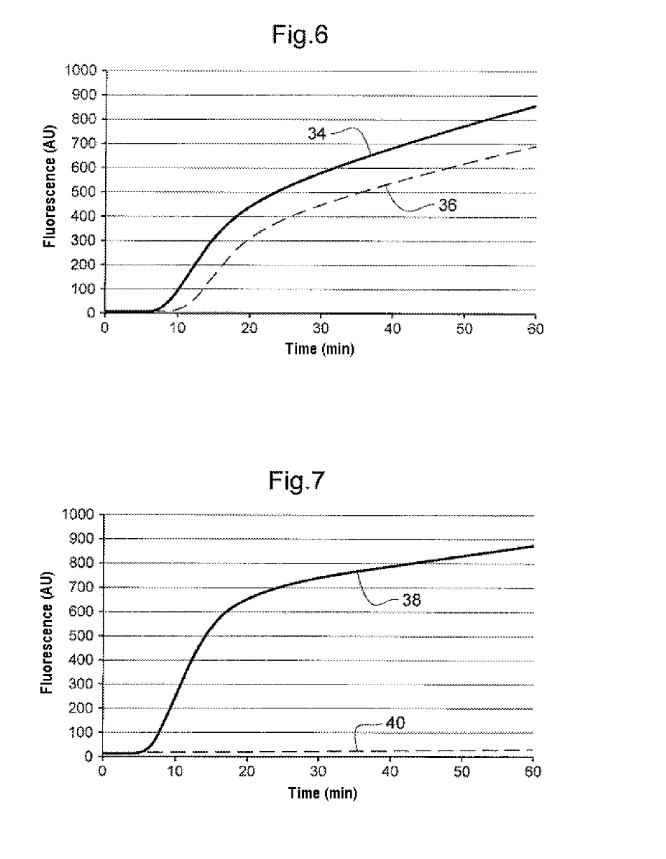

FIG. 6 is a graph showing the strength of a fluorescent signal as a function of time according to the measuring method in accordance with the invention, with a low-molecular-weight heparin; and

FIG. 7 is a graph showing the strength of a fluorescent signal as a function of time according to the measuring method in accordance with the invention, with a compound which has no effect on heparin.

In a first part of the detailed description, firstly it will be explained how the neutralization of the glycosaminoglycans, or heparins, contained in a blood sample is evaluated, and secondly, examples will be given of a proteolytic enzyme substrate of general formula: Q¹-Xaa²-Xaa¹-rhodamine₁₁₀-Q², which actually makes it possible to neutralize these glycosaminoglycans. It will be observed that one of the merits of the invention has been to bring to light the effect of glycosaminoglycan neutralization by certain proteolytic enzyme substrates. In addition, this principle of heparin neutralization will be verified for two types of heparin, unfractionated heparins, or UFHs, and low-molecular-weight heparins, or LMWHs.

In a second part of the description, certain enzyme substrates identified in the first part will be applied in a measuring method in accordance with the invention, in which, besides their neutralizing effect with respect to the heparins, they make it possible to measure the activity of the enzymes.

Before referring to the diagrams of the figures, the evaluation method used to quantify the neutralization of the glycosaminoglycans in a blood sample, and in particular a plasma, will be described. This method makes it possible to use both unfractionated heparins and low-molecular-weight heparins. In addition, the method endeavors to test the activity of factor Xa, located at the crossroads of two enzymatic cascades resulting in the formation of thrombin. Moreover, antithrombin, which is a natural inhibitor of thrombin generation, controls the reaction. As soon as it appears, factor Xa couples to its natural substrate, prothrombin, so as to form thrombin. The heparin contained in the sample forms, itself, a complex with antithrombin, which complex promotes the inhibition of factor Xa.

Thus, a chromogenic substrate based on para-nitroaniline, which factor Xa hydrolyzes, is used and the release of the para-nitroaniline, which can be measured optically, is then inversely proportional to the concentration of heparin present in the medium. A measurement of the anti-Xa activity is thus deduced therefrom.

Reference will be made to the diagram of FIG. 1, for analyzing the results of a first example implementing the abovementioned evaluation method.

In this first example, the heparin used is an unfractionated calcium heparin sold under the brand name: “Calciparine®”. According to the evaluation method, the anti-Xa activity is recorded and transferred onto the y-axis, as a function of the heparin concentration of the medium. The heparin concentrations vary between 0 and 1.67 IU/ml and are relatively close to the therapeutic concentrations. The first inclined curve 10 corresponds to a first series of measurements wherein physiological saline is added to the blood sample in addition to the para-nitroaniline-based substrate, while the second inclined curve 12 corresponds to a second series of measurements wherein a neutralizing synthetic polymer, polybrene, intended to neutralize the anticoagulant effects of the heparin, is added to the blood sample. This compound is well known for its effects on heparin and is usually employed in methods for measuring the activity of proteolytic enzymes in order to actually dispense with the anticoagulant effect of heparin.

Thus, the first curve 10 illustrates the variation in anti-Xa activity as a function of the heparin concentration in the medium, and the second curve 12 illustrates the lack of variation in anti-Xa activity, when, on the contrary, a heparin-neutralizing agent is used.

The percentage neutralization by the synthetic polymer is determined as being the ratio of the difference in the variation in anti-Xa activity in the medium containing the physiological saline and in the medium containing the neutralizing agent, between two heparin concentrations, and only the variation in activity in the medium containing the physiological saline, between the same two heparin concentrations, this ratio being multiplied by 100 in order to obtain the result as percentage neutralization.

The percentage neutralization can also be written more compactly: 100×(1−ΔA₁/ΔA₂), wherein ΔA₁ corresponds to the variation in activity in the presence of neutralizing compound and ΔA₂ to the variation in the presence of physiological saline. Thus, the parallel measurement of the variation in the presence of physiological saline makes it possible to establish the reference in each series of measurements.

In this first example of implementation, since the variation in anti-Xa activity in the presence of the neutralizing compound is substantially zero, the percentage neutralization is close to 100%.

Reference will now be made to the diagram of FIG. 2, showing the results of a second example of implementation of the abovementioned evaluation method, in which the two curves 14, 16 are comparable to those of the first example. The first curve 14 corresponds to a first series of measurements carried out under conditions similar to those of the first example, in which physiological saline is added to the blood sample. On the other hand, the second curve 16 corresponds to a second series of measurements wherein the neutralizing synthetic polymer is no longer added to the blood sample, but a proteolytic enzyme substrate in accordance with the invention of formula: R²—Xaa⁴-Xaa²-Xaa¹-rhodamine₁₁₀-Xaa³, in which, according to a first variant V(1), Xaa¹ and Xaa³ are respectively arginine and Xaa² and Xaa⁴ glycine, while R² is a protective group, is added to the blood sample. The substrate which is the subject of this first variant is written synthetically as: R²GGR-Rhod₁₁₀-R.

This proteolytic enzyme substrate obviously plays no role here in the evaluation method as tracer. This second example has no objective other than to show only the anti-heparin effect of this substrate.

The heparin used is also Calciparine®, and it is observed, in a manner analogous to that of the first example, that the substrate which is the subject of the invention has a neutralizing effect close to 100%. As will be explained in the remainder of the description, this enzyme substrate is an excellent candidate for the measuring method according to the invention, since it makes it possible to simultaneously neutralize the heparin in the medium and to measure the enzymatic activity.

The diagram of FIG. 3 shows the results of a third example of implementation of the evaluation method, in which two pairs of curves 18, 20 and 22, 24, which are respectively substantially the same, exhibit a profile comparable to that of the curves of the preceding examples. The first pair of curves 18, 20 corresponds to two first series of measurements carried out under conditions similar to those of the preceding examples, in which physiological saline is added to the blood sample. The second pair of curves 22, 24 corresponds to two second series of measurements wherein a proteolytic enzyme substrate in accordance with the invention of general formula Q¹-Xaa²-Xaa¹-rhodamine₁₁₀-Q², in which, according to a second variant V(2), Q² is Xaa¹-Xaa²-Q¹, while Q¹ is the R¹O—(C═O)—CH₂—CO group, Xaa¹ being arginine, Xaa² valine and R¹ the ethyl group, is added to the blood sample. The substrate which is the subject of this second variant is written synthetically: (EtM-VR)₂-Rhod₁₁₀.

This proteolytic enzyme substrate also does not interfere as a tracer in the evaluation method. The heparin used is also Calciparine®, and here again it is observed that the substrate which is the subject of the invention has a heparin-neutralizing effect close to 100%.

Reference will presently be made to the diagram of FIG. 4, showing the results of a fourth example of implementation of the evaluation method. A first series of measurements is carried out under conditions similar to the preceding examples, in which physiological saline is added to the blood sample, and a first curve 26, corresponding well to the first curves of the preceding examples, is obtained.

A second series of measurements is then carried out, wherein a proteolytic enzyme substrate not in accordance with the invention, but of formula: Q¹-Xaa²-Xaa¹-rhodamine₁₁₀-Q², in which Q² is an acetyl group, while Q¹ is the R¹O—(C═O)—CH₂—CO group, Xaa¹ being arginine, Xaa² valine and R¹ the ethyl group, is added to the blood sample. This substrate, written synthetically EtM-VR-Rhod₁₁₀-Ac, has, on one side of the rhodamine₁₁₀, a peptide link identical to that of the preceding example, and on the other side, an acetyl group. A second curve 28 corresponding to a second series of measurements is plotted, and it is also not substantially horizontal, but substantially parallel to the first curve 26.

It is thus observed that this enzyme substrate neutralizes heparin very little. The percentage neutralization obtained according to the formula 100×(1−ΔA₁/ΔA₂) varies between 15% and 30% depending on heparin concentrations. Thus, this substrate is a poor candidate for preparing a proteolytic enzyme substrate forming both a tracer reagent and a compound making it possible to neutralize heparins.

The results, displayed as percentage neutralization of Calciparine®, of three other embodiment variants of the enzyme substrate in accordance with the invention are reported in table I below, for four different concentrations of heparin between 0.42 IU/ml and 1.67 IU/ml.

According to a third variant V(3), Q² is Xaa¹-Xaa²-Q¹, and Xaa¹ is arginine and Xaa² and Xaa⁴ are respectively glycine, while R² is a protective group. The substrate which is the subject of this third variant is written synthetically: (R²GGR)₂-Rhod₁₁₀.

According to a fourth variant V(4), Q² is hydrogen H, Xaa¹ is arginine and Xaa² and Xaa⁴ are respectively glycine, while R² is a protective group, i.e., synthetically, R²GGR-Rhod₁₁₀-H.

According to a fifth variant V(5), Q² is the benzoic group, Xaa¹ is arginine, and Xaa² and Xaa⁴ are respectively glycine, while R² is a protective group, i.e. R²GGR-Rhod₁₁₀-Benzoic.

TABLE I

| [IU/ml] | V(3) | V(4) | V(5) |
|---|---|---|---|
| 0.42 | 90% | 96% | 100% |
| 0.83 | 93% | 90% | 97% |
| 1.25 | 94% | 89% | 98% |
| 1.67 | 96% | 80% | 98% |

It is thus observed that the enzyme substrates which are the subject of variants V(3), V(4) and V(5) exhibit a very good neutralizing effect with respect to an unfractionated heparin, Calciparine®.

Still in this first part of the description, the abovementioned evaluation method was implemented with a low-molecular-weight heparin, Fragmine®.

The results of the percentage neutralization of this heparin for the five substrate variants described above, V(1), V(2), V(3), V(4) and V(5) have been reported.

TABLE II

| [IU/ml] | V(1) | V(2) | V(3) | V(4) | V(5) |
|---|---|---|---|---|---|
| 0.42 | 64% | 71% | 88% | 83% | 77% |
| 0.83 | 65% | 78% | 90% | 83% | 75% |
| 1.25 | 64% | 79% | 91% | 72% | 75% |
| 1.67 | 72% | 78% | 91% | 64% | 74% |

This table II shows that the neutralizing effect of enzyme substrates in accordance with the invention, according to the five embodiment variants previously described, is retained with a low-molecular-weight heparin. It will be observed that the results obtained with this fractionated low-molecular-weight heparin are substantially worse than those obtained with the unfractionated heparin. However, this does not in any way compromise the use of these substrates in a method for measuring the activity of proteolytic enzymes that will be described below and by virtue of which the presence of heparins, whether unfractionated or of low molecular weight, is overcome.

Thus, the present invention also relates to a method for measuring the activity of proteolytic enzymes of a blood sample, which makes use of an enzyme substrate of general formula: Q¹-Xaa²-Xaa¹-rhodamine₁₁₀-Q², in which Xaa¹ and Xaa² are amino acids; and Q² is Xaa¹-Xaa²-Q¹, or an amino acid Xaa³, or a group comprising an aryl group, or else H. By virtue of this enzyme substrate, it is possible not only to inhibit the anticoagulant capacity of the heparins in a blood sample, but also, and simultaneously, to measure the activity of an enzyme, and this is, moreover, the advantage of the use of the substrates which are the subject of the invention.

According to one embodiment of the measuring method, the enzymatic activity monitored is that of thrombin generation. The principle lies in the monitoring of the measurement of a fluorescent signal after the enzyme substrate has been brought into contact with a blood sample containing heparin, and the reaction conditions allow the thrombin to cleave the substrate between the peptide part and the fluorophore, in this case rhodamine₁₁₀.

In order to implement the method, a blood sample of 40 μL containing heparin is first of all introduced into a microcuvette. 10 μL of the enzyme substrate in accordance with the invention mixed with an initiating reagent, and 10 μL of activating reagent are added thereto. The initiating reagent is calcium citrate and it has a concentration of 16.7×10⁻³ mol.L⁻¹. The activating reagent is tissue factor at a concentration of 10⁻¹² mol.L⁻¹.

Next, an excitation signal having a predetermined wavelength is emitted through the microcuvette and the response to the excitation signal is recorded optically over time. Thus, the greater the amount of thrombin generated, the greater the signal response.

According to one particular embodiment, the enzyme substrate chosen is that corresponding to the embodiment variant V(3) of the first part of the detailed description, and of synthetic formula (R²⁻GGR)₂-Rhod₁₁₀. In addition, the method is implemented with an unfractionated heparin and a low-molecular-weight heparin.

The results are reported in the graphs of FIGS. 5 and 6. FIG. 5 illustrates a first experiment using an unfractionated heparin, while FIG. 6 illustrates a second experiment using a low-molecular-weight heparin. The strength of the excitation-signal response is reported on the y-axis, while the time scale is represented on the x-axis.

On the graph of FIG. 5, the first upper curve 30 corresponds to a series of measurements carried out with a first blood sample not containing heparin, while the second lower curve 32 is produced with a second blood sample having a heparin concentration of 1.5 IU/ml.

The two curves 30, 32 are substantially parallel and it is quite naturally deduced therefrom that the heparin has a very small effect on the result of the thrombin activity measurement.

On the graph of FIG. 6, illustrating a second experiment, and in a manner analogous to that of FIG. 5, the first upper curve 34 corresponds to a series of measurements carried out with a first blood sample not containing heparin, while the second lower curve 36 is produced with a blood sample having a heparin concentration of 1.5 IU/ml.

Here also, the two curves 34, 36 are substantially parallel and the presence of the heparin is dispensed with in order to determine the thrombin activity.

Thus, it is understood that the enzyme substrate corresponding to the embodiment variant V(3), and as the method of evaluation described in the first part had revealed, neutralizes the effects of heparin, and furthermore here, makes it possible to measure the thrombin activity.

Represented in FIG. 7 is a graph showing the results of a third experiment wherein the substrate used according to the abovementioned measuring method is the enzyme substrate EtM-VR-Rhod₁₁₀-Ac, not in accordance with the invention. The method of evaluation above had made it possible to discard this substrate since it neutralizes heparins weakly.

The first curve 38 of the graph of FIG. 7 corresponds to a series of measurements carried out with a first blood sample not containing heparin, and the second curve 40 to a series of measurements with a blood sample containing 1.5 IU/ml of heparin.

The second curve 40 is substantially horizontal, and the strength of the signal corresponding to the thrombin activity corresponds, after 30 minutes, to substantially 3% of the strength of the signal of the series of measurements carried out without heparin. Thus, the heparin in the medium is not neutralized and there is no thrombin production.

With this type of substrate, it is impossible to measure the activity of an enzyme, and in this case thrombin, without being subjected to the impact of the heparin.

Moreover, with the substrates according to the four other variants, results similar to those obtained with the enzyme substrate corresponding to the embodiment variant V(3) are obtained.

The present invention also relates to a kit for measuring the activity of proteolytic enzymes of a blood sample, for implementing the measuring method described above.

According to the invention, the kit comprises a substrate according to one of the variants V(1) to V(5). Of course, it could comprise a substrate according to another variant defined by the abovementioned general formula. The proteolytic enzyme substrate, at a concentration of 300×10⁻⁶ mol.L⁻¹, is then mixed with calcium citrate at a concentration of 16.7×10⁻³ mol.L⁻¹, in a first container. This mixture forms a first reagent. Tissue factor at a concentration of 10⁻¹² mol.L⁻¹ is contained in another container and constitutes the second reagent.

These two reagents are then capable of being used in a microcuvette, with the blood sample, at the time the enzymatic activity is measured.

1. A method of inhibiting, in a blood sample containing a glycosaminoglycan, the anticoagulant capacity of said glycosaminoglycan, comprising applying a proteolytic enzyme substrate of general formula: Q¹-Xaa²-Xaa¹-rhodamine₁₁₀-Q², in which:
Xaa¹ and Xaa² are amino acids; and
Q² is Xaa¹-Xaa²-Q¹, or an amino acid Xaa³, or a group comprising an aryl group, or H.

2. The method of claim 1, wherein in the proteolytic enzyme substrate Q¹ is:
H; or
the R¹O—(C═O)—CH₂—CO group in which R¹ comprises an alkyl or aryl group; or
the R²—Xaa⁴ group in which Xaa⁴ is an amino acid and R² a protective group or H.

3. The method of claim 2, wherein in the proteolytic enzyme substrate wherein Xaa¹ is a basic amino acid.

4. The method of claim 3, where in the proteolytic enzyme substrate Xaa¹ is arginine.

5. The method of claim 4, wherein in the proteolytic enzyme substrate Q² is Xaa¹-Xaa²-Q¹, while Q¹ is the R²—Xaa⁴ group, Xaa² and Xaa⁴ being respectively glycine, so as to confer on said substrate a low affinity with respect to proteolytic enzymes.

6. The method of claim 4, wherein in the proteolytic enzyme substrate Q² is Xaa¹-Xaa²-Q¹, while Q¹ is the R¹O—(C═O)—CH₂—CO group, Xaa² being valine and R¹ the ethyl group, so as to confer on said substrate a low affinity with respect to proteolytic enzymes.

7. The method of claim 1, wherein in the proteolytic enzyme substrate wherein Xaa³ is arginine.

8. The method of claim 1, wherein in the proteolytic enzyme substrate when Q² is a group comprising an aryl group, Q² comprises a —(C═O)ₘ—(O)ₙ— group linked to rhodamine₁₁₀, in which n=0 or 1, and m=0 or 1.

9. The method of claim 8, wherein in the proteolytic enzyme substrate n=0 and m=1.

10. The method of claim 9, wherein in the proteolytic enzyme substrate Q² is the benzoyl group.

11. The method of claim 9, wherein in the proteolytic enzyme substrate Q² is the 2-phenylbutanoyl group.

12. The method of claim 8, wherein in the proteolytic enzyme substrate Q² also comprises a heteroatom.

13. Method for measuring the activity of proteolytic enzymes of a blood sample, comprising:
providing a blood sample which contains proteolytic enzymes and which may contain a glycosaminoglycan;
providing a proteolytic enzyme substrate capable of providing a signal when said proteolytic enzymes react with said substrate;
bringing said blood sample into contact with said proteolytic enzyme substrate, while the anticoagulant capacity of said glycosaminoglycan is inhibited so as to allow said proteolytic enzymes to react freely with said substrate in order to provide a signal representative of the activity of the proteolytic enzymes in said sample;
using a proteolytic enzyme substrate according to claim 1, so as to both inhibit the anticoagulant capacity of the glycosaminoglycan and to measure the activity of the proteolytic enzymes, with a single compound.

14. Measuring method according to claim 13, further comprising, providing an activating reagent which makes it possible to induce the generation of proteolytic enzymes, and bringing said activating reagent into contact with said blood sample and said proteolytic enzyme substrate.

15. Measuring method according to claim 14, further comprising electing said activating reagent from the group consisting of tissue factor, phospholipids, thromboplastin, kaolin, ellagic acid, collagen, adenosine diphosphate, arachidonic acid, a thrombin receptor-activating peptide, or a combination thereof.

16. Measuring method according to claim 13, further comprising providing an initiating reagent for initiating the proteolytic enzyme reactions, and bringing said initiating reagent into contact with said blood sample and said proteolytic enzyme substrate.

17. Measuring method according to claim 16, wherein said initiating reagent is calcium.

18. Measuring method according to claim 13, wherein said blood sample is brought into contact with said proteolytic enzyme substrate at a substrate concentration of between 50×10⁻⁶ and 1000×10⁻⁶ mol.L⁻¹.

19. A kit for measuring the activity of proteolytic enzymes of a blood sample, for implementing the method according to claim 13, to inhibit, in a blood sample containing a glycosaminoglycan, the anticoagulant capacity of said glycosaminoglycan, the kit comprising:
an activating reagent for inducing the generation of proteolytic enzymes;
a proteolytic enzyme substrate of general formula: Q¹-Xaa²-Xaa¹-rhodamine₁₁₀Q², in which:
Xaa¹ and Xaa² are amino acids; and
Q² is Xaa¹-Xaa²-Q¹, or an amino acid Xaa³, or a group comprising an aryl group, or H; and
an initiating reagent for initiating the enzymatic reactions.

20. A measuring kit according to claim 19, wherein said proteolytic enzyme substrate is mixed with said initiating reagent.

21. A measuring kit according to claim 19, wherein said activating reagent is mixed with said initiating reagent.
